(19) United States
(12) Patent Application Publication
(10) Pub. No.: US 2008/0015442 A1
(43) Pub. Date: Jan. 17, 2008

(54) Ultrasound Waveguide

(76) Inventors: Malcolm John Watson, Glasgow (GB); George A. Corner, Kirrintillock (GB); Katherine J. Kirk, Glasgow (GB); Alexander Cochran, Glasgow (GB); David Ian Arthur Lines, Edinburgh (GB); Srinath Rajagopal, Karnataka (IN)

Correspondence Address: BURNS & LEVINSON LLP, 125 Summer Street, Boston, MA, 02110-1624, US

(21) Appl. No.: 11/629,568

(22) PCT Filed: Jun. 16, 2005
(86) PCT No.: PCT/GB05/02400

§ 371 Date: Sep. 19, 2007

(30) Foreign Application Priority Data
Jun. 16, 2004 (GB) 0413382.3
Jul. 22, 2004 (GB) 0416370.5

(51) Int. Cl. A61B 8/14

(52) U.S. Cl. 600/459

(57) ABSTRACT

An ultrasound waveguide that is attachable to an ultrasound probe so as to identify a target area on a target object. The ultrasound waveguide has an ultrasound transducer coupling signal to be transmitted through a guide means. The ultrasound waveguide also has a positioning means for positioning the guide means in relation to the target area on the target object. The guide means is provided with a channel that provides a discontinuity within the guide means that causes a discontinuity in the ultrasound signal emitted by the probe. The presence of this discontinuity allow for proper alignment of the ultrasound waveguide with the target object.

The present invention relates to the field of ultrasound waveguides and in particular to the application of an ultrasound waveguide, employed in conjunction with an ultrasound transducer, within the field of ultrasonography.

Ultrasonography is used in a variety of medical diagnosis and examination applications. These include the detection of malignant and benign tumours, providing images of foetuses for assessment of their development, and monitoring blood flow within various vital organs and foetuses. A variety of ultrasonographic techniques have been developed for such applications.

It is known to those skilled in the art that there is often a need for the expeditious and accurate location of a needle insertion position on a patient by a clinician. An example of such an occasion occurs when there is a need to provide a patient with a local anaesthetic in the sub-arachnoid or epidural space region, either directly or via a catheter. The purpose of such an injection may be to provide analgesia to the patient. Alternatively, the anaesthetic may be administered to provide a sufficient loss of sensation in the patient to enable particular types of surgical procedure to be carried out. Particular examples of such procedures include:
Obstetric surgery, such as trial of forceps, caesarean section (emergency or elective), manual removal of retained products of conception, repair of third degree perineal tear
a Lower limb orthopaedic surgery, such as hip, knee or ankle replacements
Gynaecological surgery, such as hystectomy, oophectomy, or pelvic clearance for neoplasm
General surgery, such as panproctocolectomy, Hartmanns procedure, gastectomy, Whipple's procedure
Cardiothoracic surgery, such as coronary artery bypass grafting, valve replacement, pneumonectomy, pleurodiesis
Transplant surgery, such as cardiac, hepatic, lung or renal transplants

This type of anaesthetic is referred to as a central neuroaxial block.

In order to administer effectively the anaesthetic into the epidural space, it is necessary to correctly identify a safe lumbar interspace. At present, clinicians rely on three main techniques to locate a lumbar interspace. The first is based on an assumption that an imaginary line joining the iliac crests crosses close to the 4^{th} lumbar spine. However, in practice this line may in fact cross the spine cord higher or lower than the 4^{th} lumbar spine.

Secondly, medical students are taught that the spinal cord ends at L₁₋₂. In actuality, it is known that the position of the end of the spinal cord follows a normal distribution, with the mean position at L₁₋₂ It has been shown that the spinal cord ends opposite the body of L₃ in 1-3% of cases, with increased variance in women patients.

A further technique employs a reliance on a lack of paraesthesia in the region, a reliance which research has shown to be misplaced.

Additional techniques include the inherently unreliable manual detection by the anaesthetist, as well as x-ray imaging techniques, which are unsuitable for use on women during pregnancy.

In addition to the inherent disadvantages of the above techniques, further problems are created when attempting to locate the lumbar inter-space on certain groups of patients. Difficult patients include patients with anatomical abnormalities, which may be congenital (e.g. scoliosis) or acquired (e.g. surgical fusion of lumbar spinous processes following lumbar disc prolapse).

Problems are also encountered with obese patients, where excessive subcutaneous tissue prevents the palpation of subcutaneous landmarks.

Patients that have been subject to several previous failed insertion attempts also pose problems for an anaesthetist. A further example is in the case of a patient that has a coagulopathy or thrombocytopenia. In this situation it is important to insert the needle with minimal trauma, and to reduce the risk of bleeding complications.

The present invention identifies the drawbacks of the established techniques and procedures, and proposes to utilise ultrasound to assist in the location and identification of anatomical features. The specific description is written in the context of administering an anaesthetic to a patient. However, it will be appreciated by those skilled in the art that the methods and apparatus described apply equally to the location or identification of various anatomical features of a patient for any purpose. Furthermore, the techniques apply equally to the alignment of catheters.

It is an aim of at least one aspect of the invention to provide apparatus to aid in the location of a target area on a patient.

It is an aim of at least one aspect of the invention to provide a method of locating target areas on a patient with improved accuracy, speed, and effectiveness.

It is an aim of at least one aspect of the invention to provide a method and apparatus for identifying a lumbar interspace on a patient.

It is an aim of at least one aspect of the invention to provide an improved method of aligning a needle or catheter with a lumbar interspace of a patient.

Further aims and objects of the invention will become apparent from reading the following description.

### SUMMARY OF INVENTION

According to a first aspect of the present invention there is provided an ultrasound waveguide for coupling with an ultrasound transducer so as to provide a means for identifying a target area on a target object, the ultrasound waveguide comprising an ultrasound transducer coupling means, a guide means and a positioning means for positioning the guide means in relation to the target area on the target object.

Preferably, the positioning means comprises an anterior face contactable with a surface of the target object and a posterior face comprising a reflecting section for reflecting an ultrasound field generated by the ultrasound transducer so as to exit the ultrasound waveguide through the anterior face.

Preferably, the anterior face is planar.

Preferably, the ultrasound transducer coupling means is shaped to receive the ultrasound transducer.

Optionally, the ultrasound transducer coupling means further comprises a fastening means for maintaining an acoustic contact between the ultrasound transducer and the ultrasound transducer coupling means.

Preferably, the fastening means is selected from a group comprising a set of clips, nuts and bolts, a frame, tape and a hollow located within the shaped surface.

Preferably, the ultrasound transducer coupling means is provided with a shaped surface that is shaped to conform to the shape of the ultrasound transducer.

Preferably, the shaped surface is arcuate.

Preferably, the guide means is provided with a channel that provides a discontinuity within the guide means that causes a discontinuity in the ultrasound signal emitted by the probe.

The channel may be shaped to minimise acoustic artefacts produced by an ultrasound signal.

Preferably an acoustic absorber is included in the channel.

Optionally, the channel extends from the reflecting section of the posterior face through to the anterior face.

Preferably, the channel comprises a recess located on an edge of the positioning means.

Alternatively, the channel is enclosed by the positioning means.

The channel may be at least partially defined by a first side wall and a second side wall, the first and second side walls being inclined with respect to the normal to the anterior face such that the channel has a first width at the posterior surface and a second width at the anterior surface.

Preferably, the first width at the posterior surface is greater than the second width at the anterior surface.

Optionally, the channel is further defined by an internal lateral side wall that is parallel to the normal to the anterior surface.

Preferably, the internal side wall comprises a groove the sides of which are non parallel to the shaped surface suitable for receiving the ultrasound transducer.

Optionally, the groove is V-shaped.

Alternatively, the guide means comprise a pair of guide members protruding from the reflecting section of the posterior face.

Preferably, the guide means is adapted to receive a needle.

The guide means may be sized to allow the needle to be redirected following initial penetration of the target object.

Preferably, the guide means is inhomogeneous such that the acoustic impedance of the guide means is variable.

Optionally, the guide means is provided with layers of material at least some of which have different acoustic impedances.

Preferably, the guide means is made from a material with an acoustic impedance to match that of the target object.

Preferably, the material is a tissue mimicking material.

Preferably, the guide means comprises a gel.

Optionally, the ultrasound wave guide further comprises a support structure for supporting the guide means.

The support structure may be used to increase the accuracy of the identification of the target area.

Preferably, the support structure is a shell adapted to enclose the guide means.

Preferably, the support structure is an external frame.

More preferably, the support structure further comprises an acoustic absorber lining.

Optionally, the support structure comprises reinforcing threads extending through the guide means.

Preferably, the ultrasound probe further comprises a sheath that provides a sterile barrier between the probe and the target object.

Preferably, the sheath envelops the ultrasound transducer.

Alternatively, the sheath envelops both the ultrasound transducer and the ultrasound waveguide.

Optionally the sheath is integrated directly with the ultrasound waveguide.

Preferably, the target object is a human body.

More preferably the target object is the lumbar region of a human body.

According to a second aspect of the present invention, there is provided an ultrasound probe for identifying a target area on a target object, the ultrasound probe comprising an ultrasound transducer and an ultrasound waveguide as defined with reference to the first aspect of the invention.

According to a third aspect of the present invention, there is provided apparatus for identifying a target area on a patient, comprising an ultrasound probe in accordance with the second aspect of the present invention and a display for displaying an image produced in response to a signal generated by the ultrasound probe.

Most preferably the image enables identification of the target area.

Optionally the image displays the location of the target area in relation to the guide means.

According to a fourth aspect of the present invention, there is provided a method of identifying a target area on a target object, the method comprising the steps of:

positioning an ultrasound probe in relation to the target object, the ultrasound probe having an ultrasound waveguide and guide means coupled to an ultrasound transducer;

displaying an image of the target object;

identifying a target area from said image based on an image artefact created by the guide means; and

positioning the guide means in relation to said target area.

Preferably, the target object is a human body.

More preferably, the target object is the lumbar region of a human body.

Optionally the method includes the additional step of aligning the guide means with the target area.

The method may include the additional step of positioning a needle within the guide means, such that the needle is positioned with respect to the target area.

The method may include the additional step of repositioning the needle within the guide means, such that the needle is positioned with respect to the target area.

The method may include the additional step of marking the target area on the target object.

The method may include the additional step of displaying an image of the needle in relation to the target object.

Preferably, the target area is a lumbar interspace of a patient, and the guide means is positioned in relation to said lumbar interspace.

The method may include the additional step of positioning a needle with respect to the guide means, such that the needle is positioned with respect to the lumbar interspace.

The method may include the additional step of aligning the guide means with the lumbar interspace.

The method may include the additional step of directing the displayed image of the needle towards the target object.

The method may include the additional step of marking a target area corresponding to the lumbar interspace.

According to a fifth aspect of the invention, there is provided a method for inserting a needle into a lumbar interspace of a patient, the method comprising the steps of:

positioning an ultrasound probe in relation to the lumbar region of the body of the patient, the ultrasound probe having an ultrasound waveguide and guide means coupled to an ultrasound transducer;

displaying an image of the lumbar region;

identifying a lumbar interspace from said image; positioning the guide means in relation to said lumbar interspace based on an image artefact created by the guide means; and

inserting a needle into the lumbar region of the patient via the guide means.

The method may include the additional step of aligning the guide means with the lumbar interspace.

The method may include the additional step of displaying an image of the needle in relation to the target object.

The method may include the additional step of marking a target area corresponding to the lumbar interspace.

### DETAILED DESCRIPTION

Aspects and advantages of the present invention will become apparent upon reading the following detailed description and upon reference to the following drawings in which:

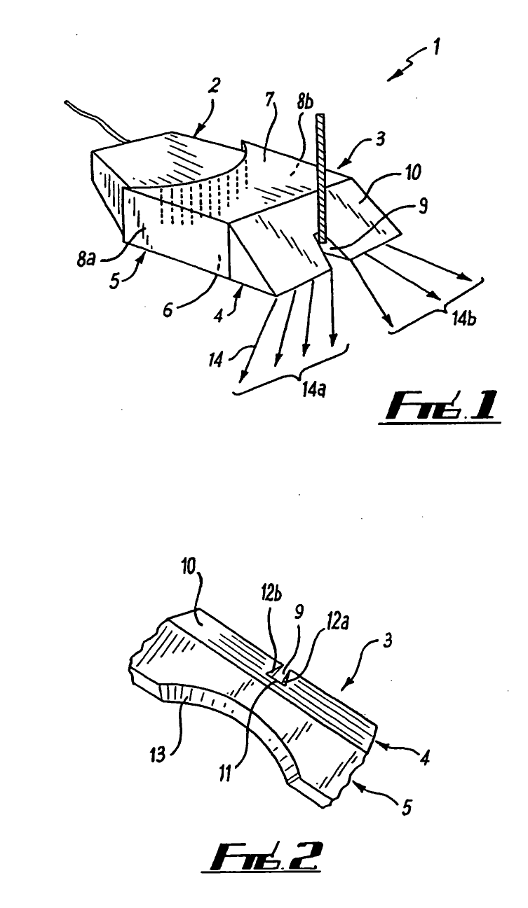

FIG. 1 shows a perspective view of an ultrasound probe in accordance with an aspect of the present invention;

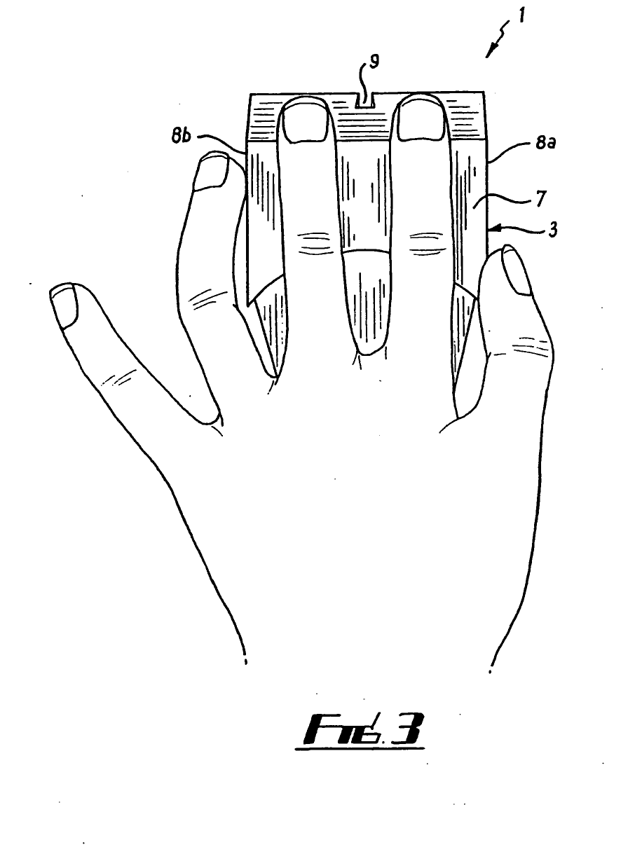

FIG. 2 shows a perspective view of an ultrasound waveguide employed within the ultrasound probe of FIG. 1 in accordance with an alternative aspect of the present invention;

FIG. 3 shows an example of how an operator holds the ultrasound probe of FIG. 1;

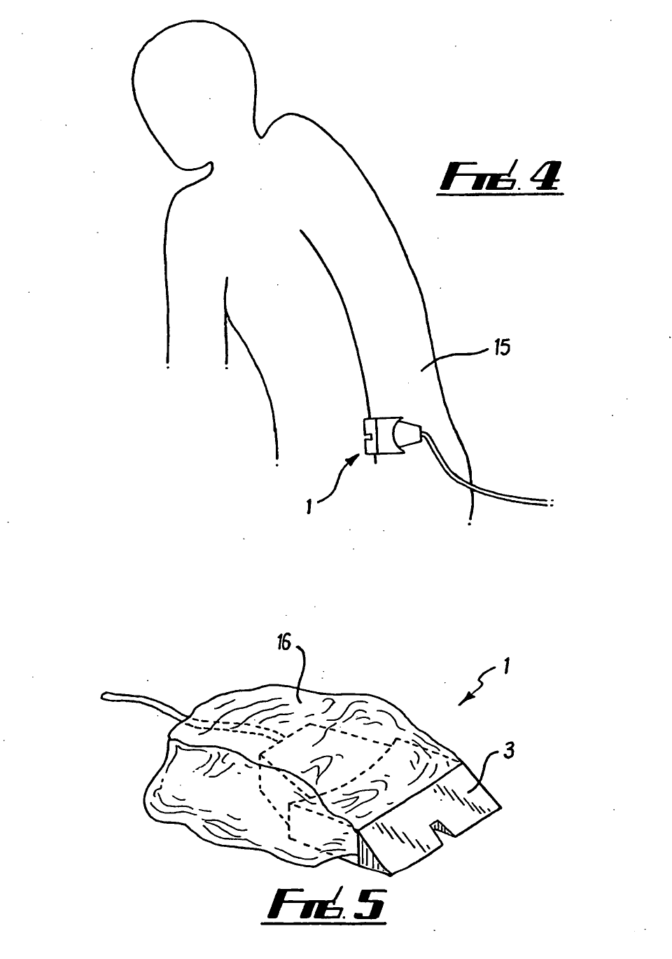

FIG. 4 shows an example of how the ultrasound probe of FIG. 1 is positioned on a patient;

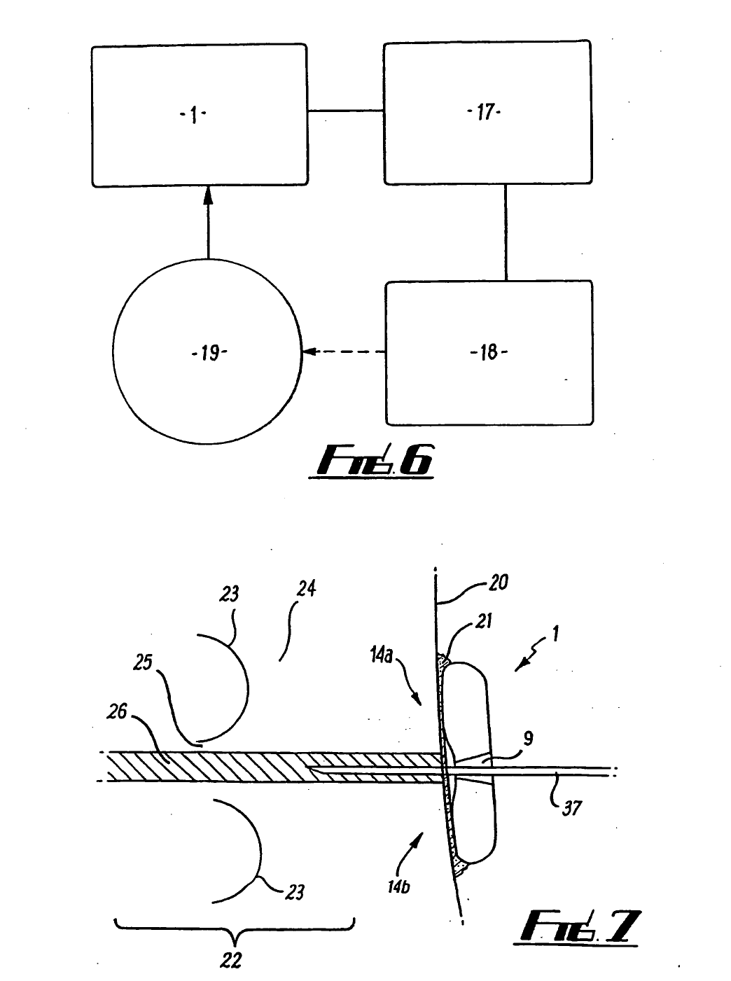

FIG. 5 shows a perspective view of the ultrasound probe of FIG. 1 deployed in conjunction with a sterile sheath;

FIG. 6 shows a schematic overview of a system in accordance with a further alternative aspect of the present invention;

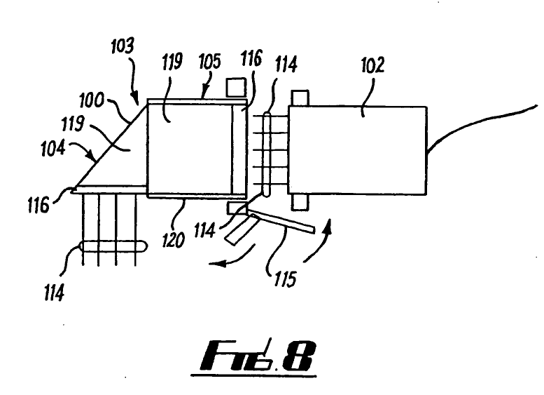

FIG. 7 shows an example of an image produced by the system of FIG. 6;

FIG. 9 shows a plan view of an alternative embodiment of the ultrasonic waveguide;

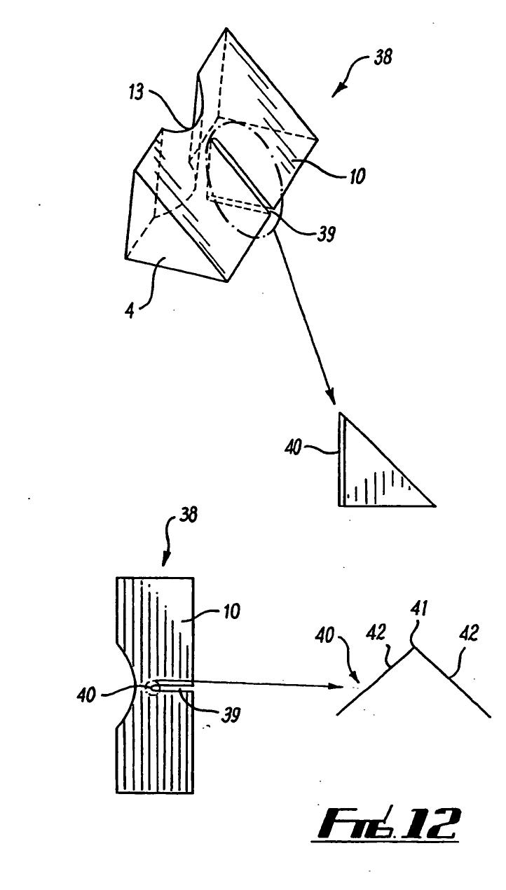

FIG. 10 shows a plan view of a further alternative embodiment of the ultrasonic waveguide;

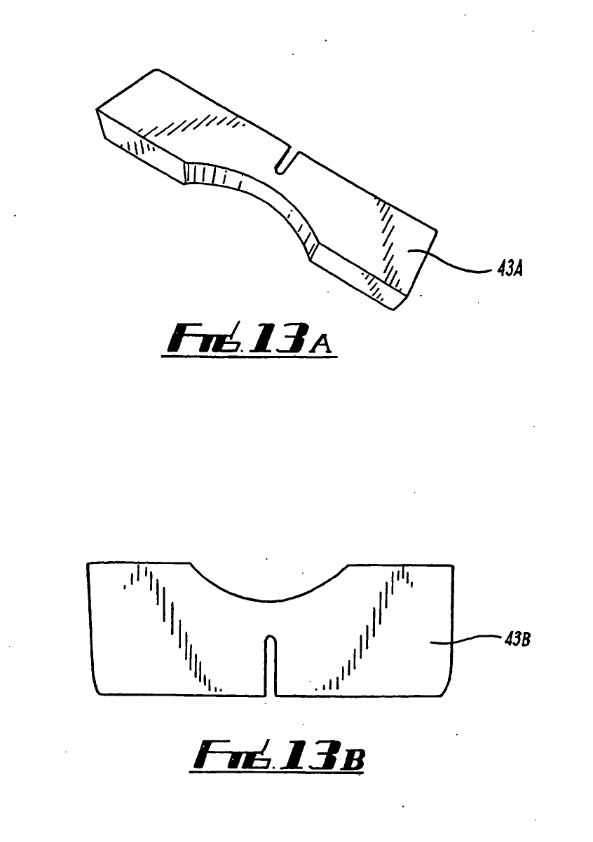

FIG. 11 shows a plan view of a yet further alternative embodiment of the ultrasonic waveguide; and

FIG. 12 shows a perspective view of a yet further alternative embodiment of the ultrasonic waveguide;

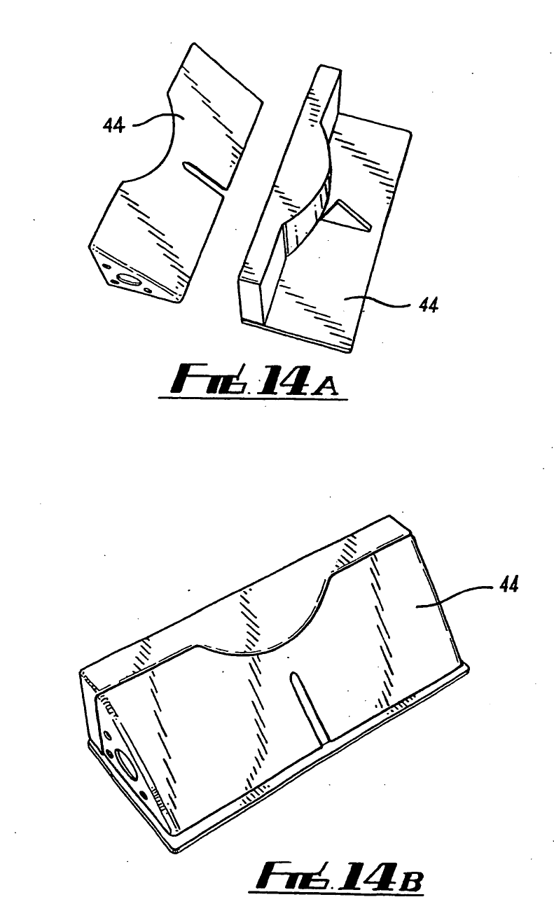

FIG. 13 shows waveguides formed of a tissue mimicking material according to a yet further alternative embodiment of the ultrasonic waveguide;

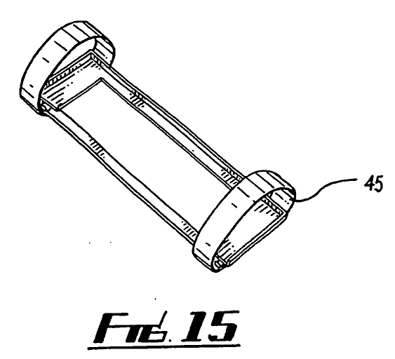

FIG. 14 shows a mould suitable for use in forming the waveguides shown in FIG. 13; and

FIG. 15 shows a frame suitable for supporting the waveguides shown in FIG. 13.

FIG. 1 is a perspective view of an ultrasound probe 1 in accordance with an aspect of the present invention. The ultrasound probe 1 comprises a standard ultrasound transducer 2, as commonly employed by those skilled in the art of ultrasonography, and an ultrasound waveguide 3. FIG. 2 is a perspective view of the ultrasound waveguide 3 in isolation.

From FIGS. 1 and 2 the ultrasound waveguide 3 can be seen to comprise two distinct sections, namely a right angled isosceles prism section 4 and a substantially cuboidal prism section 5. Both the right angled. isosceles prism section 4 and the cuboidal prism section 5 are made from a material with an acoustic impedance chosen to match that of the target object, in this case the material being Rexolite. The sections 4 and 5 may be made from a single piece of material. The two sections are integrated as a single acoustic prism so as to provide a substantially planar anterior face 6. For clarity purposes the face of the ultrasound waveguide 3 opposite the planar anterior face 6 is referred to herein as the posterior face 7. Those faces perpendicular to both the planar anterior face 6 and posterior face 7 are referred to as the lateral faces 8a and 8b, respectively.

The ultrasound waveguide 3 can be further seen to comprise a channel 9 extending from a hypotenuse face 10 of the right angled isosceles prism section 4 through to the planar anterior face 6. A rear wall 11 of the channel 9 (i.e. that located opposite to the open side of the channel 9) is perpendicular to the planar anterior face 6. Side walls 12a and 12b of the channel 9 are tapered so that the channel 9 formed has a narrower width at the planar anterior face 6 than at the hypotenuse face 10.

In the present invention, the channel is shaped to provide a suitable discontinuity in the transmitted ultrasound signal.

From FIGS. 1 and 2 it can also be seen that the face of the cuboidal prism section 5 located opposite to the right angled isosceles prism section 4 comprise an arcuate recess 13. The function of the arcuate recess is to receive and secure the ultrasound transducer 2. Fastening means (not shown) in the form of clips, nuts and bolts, a frame, tape and/or a hollow within the surface of arcuate recess 13 can also be employed to further secure the ultrasound transducer 2 to the ultrasound waveguide 3.

In the presently described embodiment the ultrasound transducer 2 comprises a curved transducer array employed to generate and subsequently detect ultrasound. Ultrasound waves 14 generated by the transducer 2 are coupled into the waveguide 3 at the arcuate recess. These waves 14 then travel through the waveguide 3 before being reflected at the internal surface of hypotenuse face 10 so as to exit the waveguide 3 via the planar anterior face 6. It should be noted that due to the presence of the channel 9 a discontinuity is created in the emitted ultrasound waves 14 which are split into two distinct signals 14a and 14b, respectively.

FIGS. 3 and 4 show how the ultrasound probe 1 comprising the transducer 2 and the waveguide 3 may be held against the body of the patient 15 during use. In practice a first estimate of the approximate level of the probe position can be obtained by counting interspinous spaces from the continuous echogenic signal of the sacrum. In particular, FIG. 4 shows the orientation of the probe 1 with respect to the patient's body. The planar anterior face 6 is placed flat against the lumbar region of the patient's back. The patient 15 is placed in a sitting position, with the lumbar spine flexed. The probe 1 is coated with gel and covered with a sterile sheath 16 that fixes to the ultrasound waveguide 3 (as shown in FIG. 5). In use, gel is also placed between the sterile sheath 16 and the patient's back in order to improve acoustic contact between the probe 1 and the patient's skin. The gel also enables an operator to manoeuvre the probe on the patient's back more effectively (as described in detail below).

The design of the ultrasound waveguide 3 is such that ultrasound waves 14 generated by the ultrasound transducer 2 are reflected through 90° from their plane of incidence. From the law of conservation of energy the reflected and transmitted ultrasound at any interface is given by:
Tᵢ+Rᵢ=1. (1)
where

Rᵢ=Relative intensity of reflected ultrasound energy, and

Tᵢ=Relative intensity of transmitted ultrasound energy.

For non-normal incidence Tᵢ and Rᵢ is given by:

$$\begin{matrix}{T}_{i} = 1 - {R}_{i} = \frac{4 {z}_{1} {z}_{2} cos {θ}_{1} cos {θ}_{2}}{{( {z}_{1} cos {θ}_{2} + {z}_{2} cos {θ}_{1} )}^{2}} & ( 2 ) \\ {R}_{i} = {( \frac{{z}_{1} cos {θ}_{2} - {z}_{2} cos {θ}_{1}}{{z}_{1} cos {θ}_{2} + {z}_{2} cos {θ}_{1}} )}^{2} & ( 3 )\end{matrix}$$

where,

θ₁ is the angle of incidence, and

θ₂ is the angle of reflection.

Employing these equations to ultrasound waveguide 3 provide a theoretical value for the total energy transmitted from the transducer to the patient of 99.88%.

Thus, the ultrasound waveguide 3 can be seen to be a highly efficient means for directing the ultrasound waves 14.

In an alternative embodiment the sterile sheath 16 is formed as an integral component of the waveguide 3. When the probe is deployed gel is then located both on the inside and outside of the sterile sheath 16. Within a further alternative embodiment the sterile sheath 16 is located around the ultrasound transducer 2 so that the attachment of the waveguide 3 to the ultrasound transducer 2 also acts to secure the sterile sheath 16. In this embodiment gel is required to be deployed between the ultrasound transducer 2 and the sterile sheath 16, the sterile sheath 16 and the waveguide 3 and the waveguide 3 and the patient 15.

FIG. 3 shows how the probe 1 may be held by an operator by pressing the index finger and middle fingers against the posterior face 7, with the planar anterior planar face 6 against the back. The lateral faces 8a and 8b of the ultrasound waveguide 3 are thus oriented in the saggital plane of the patient 15 and are held between the thumb and ring finger of the operator. The ulnar borders of the operator's hand can also be employed to further secure the probe 1 in the correct position. It should be noted that the shape of the probe 1 enables the operator to keep their fingers clear of the channel 9.

FIG. 6 shows schematically an arrangement of the apparatus in accordance with an aspect of the present invention. The system includes the ultrasound probe 1, a processing module 17 and a display 18. The ultrasound probe 1 is of the type shown in FIGS. 1 or 2, and communicates with the processing module 17 via the ultrasound transducer 2. The processing module 17 processes a detection signal from the ultrasound probe 1, and creates an image on display 18. It should be noted that the processing module 17 and the display 18 could simply comprise those components normally present within a standard ultrasonic imaging scanner.

In use an operator 19 views the image on the display 18, and controls the position of the probe 1 with respect to the patient 15. This causes the detection signal to change, and thus the image displayed on the display 18 as the probe 1 is held over a different part of the lumbar region.

Typically, the ultrasound transducer 2 will be operated at a frequency in the range of 2,000 kHz to 10,000 kHz, chosen to allow maximal tissue penetration and tissue spatial resolution. Use of the range of 200 kHz to 7,000 kHz also allows optimal differentiation of bone and soft tissue, in contrast to the requirements of established ultrasound techniques. This is lower than the frequency ranges typically used in ultrasonographic diagnosis applications. However, in certain applications, frequencies of up to 10,000 kHz (the frequency normally used for muscular skeletal imaging) and above may be useful signal processing techniques such as harmonic imaging can also be employed to improve the differentiation between tissue and bone areas of a patient.

The shape of the ultrasound waveguide 3 causes an image to be formed with a shadow or “blind-spot”. This corresponds to the location of the channel 9 within the waveguide 3.

An example image is shown in FIG. 7. The probe 1 is shown, pressed against contact with the skin 20 of the patient 15 via gel 21. The ultrasound, waves 14, split into two components 14a and 14b produces an image of region 22. The image shows spinous processes 23 differentiated from soft tissue 24. The image allows the operator 19 to identify the target area, which in this case is a lumbar interspace 25.

The spatial separation of the ultrasound wave components 14a and 14b causes a discontinuity in the image, shown as a shadow 26. In use, the shadow 26, and hence the channel 9, is aligned with the lumbar interspace 25.

The correspondence of the shadow 26 in the image with the channel 9 allows the operator 19 to use the channel 9 as a guide for the subsequent insertion of a needle. In use, the operator 19 positions a touchy needle centrally within the channel 9, and inserts the needle into the patient 15. The needle is aligned with a lumbar interspace 25, and passes safely through this gap into the epidural space. The needle is then used to administer the anaesthetic to the patient 15, as appropriate.

With the above-described system, the operator 19 inserts the needle into the patient 15 while visually monitoring the position of the probe 1 and needle via the display 18. The needle may be guided with the index finger and middle finger of the probe-supporting hand. Alternatively, the operator 19 may guide the needle with one hand (the dominant hand) while holding the probe 1 with the other.

The arrangement described allows the point of skin entry to be directed accurately towards the required interspace, without the need for multiple insertions. The arrangement also allows the measurement of data pertaining to anatomical parameters of the interspinous space. This includes the estimated measurement of depth of the sub-arachnoid space and epidural space and angulation of spinous interspace and size of interspace.

This provides valuable information to aid administration of the block.

It will be appreciated that the above-described technique could be used for placing alignment marks onto the skin for information purposes, or for later administration of anaesthetic.

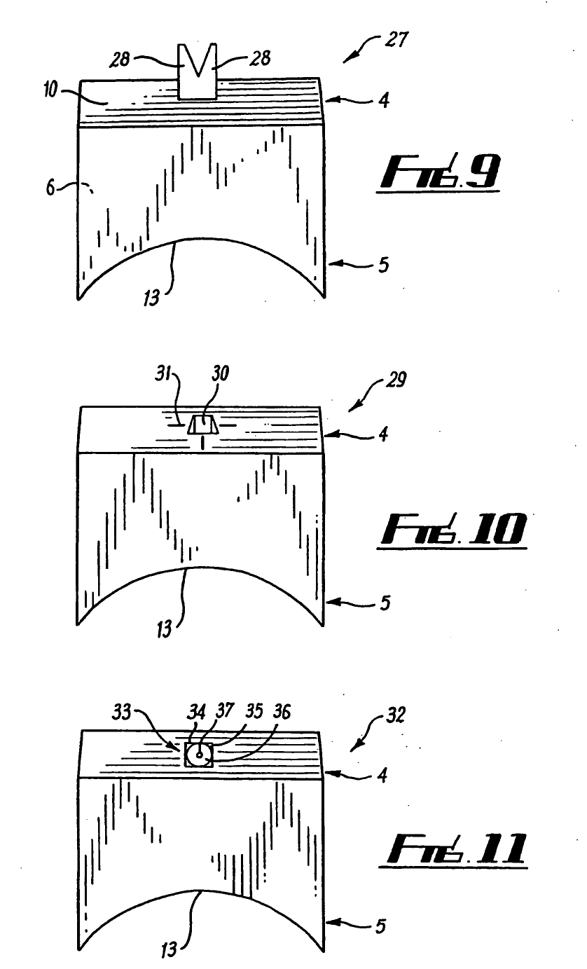

FIG. 8 shows an alternative embodiment of the present invention. The prism sections 104 and 105 comprise A shell or frame 120 that contains the wave guide material 116 and 119 and provides the means for fixing the wave guide material (consistency of jelly) to the transducer.

The wave guide material adjacent to the transducer 116 (at the coupling face) may be more fluid like and less solid than the remainder of the wave guide material 119. This inhomogeneity will, in this example allow for better acoustic contact and negate the need for acoustic gel to enhance the acoustic contact. Accordingly, the wave guide material may be inhomogeneous throughout its substance with area to make contact with the patent or transducer more fluid like (softer) improving acoustic contact

It should also be noted that the interface between the wave guide material and the fluid wave guide material should consist of a graduated change to avoid an ‘acoustic interface’ which would affect the final image

The coupling means 115 is designed to securely and firmly clasp the transducer to the ultrasound waveguide of the present invention in order to provide a good acoustic contact to optimise transmission of acoustic waves 114 through the waveguide.

The shell 120 may also include an acoustic absorber lining between the frame and the wave guide material to reduce artefacts caused by reflection of ultrasound wave s within the wave guide. For improved acoustic performance, the dimensions of the wave guide should be at least as high and broad as the transducer array to which it attaches.

In another example of the invention, the frame may consist of a lattice work of threads throughout the substance of the wave guide material instead of a shell like surface. The lattice work throughout the substance of the wave guide material will provide tensile strength for the wave guide to allow
1 attachment of the transducer via the coupling mechanism
2 strength to allow the wave guide to be use clinically without ‘falling apart’

Referring now to FIG. 9, an ultrasound waveguide 27 in accordance with an alternative embodiment of the invention is shown. The ultrasound waveguide 27 again comprises two distinct sections, namely a right angled isosceles prism section 4 and a substantially cuboidal prism section 5, from a material with an acoustic impedance chosen to match that of the target object, in this case the material again being Rexolite. The two sections are integrated as a single prism so providing a substantially planar anterior face 6. The face of the cuboidal prism section 5 located opposite to the right angled isosceles prism section 4 comprise an arcuate recess 13, the function of which is to receive and secure the ultrasound transducer 2, as previously described.

The hypotenuse face 10 of the ultrasound waveguide 27 is provided with a pair of protruding guide members 28. The anterior edges of the guide members 28 lie flush with the planar anterior face 6, and the guide members extend part way across the depth of the waveguide 27 from the anterior face 6 towards the posterior face 7. The outer faces of the guide members 28 are orientated so as to protrude orthogonally from the main body of the waveguide 27 and are parallel to one another. The inner edges are angled away from the outer edges such that an inverted v-notch is formed between the guide members 28.

The waveguide 27 can be incorporated with the ultrasound transducer 2, so as to produce an ultrasound probe, in a similar manner to that described above. The ultrasound probe is then employed in a similar manner as described in detail in relation to FIG. 3-7. Ultrasonic waves are directed anteriorly from the probe, such that an image is captured of a region of the patient's lumbar region that lies beneath the guide members 28. The image produced will be such that the point of skin entry lies at the upper region of the vertically orientated image.

In use, the operator 19 positions a touchy needle between the guide members 28, and inserts the needle into the skin. The image displayed to the operator 19 includes the needle, and the interspinous space anterior from the probe. The operator 19 is able to alter the caudal and cranial orientation of the needle, as required, so that the needle is directed safely into the epidural space. The needle is then used to administer the anaesthetic to the patient 15, as required.

The needle may be guided with the index finger and middle finger of the probe-holding hand. Alternatively, the operator 19 may guide the needle with one hand (the dominant hand) while holding the probe with the other.

Referring now to FIG. 10, an ultrasound waveguide 29 in accordance with an alternative embodiment of the invention is shown. This embodiment is similar to the embodiment shown in FIGS. 1 and 2 and can be seen to comprise the common features of the right angled isosceles prism section 4, the substantially cuboidal prism section 5 and the arcuate recess 13. However, the ultrasound waveguide 29 differs in that a channel 30 is provided in a central region of the isosceles prism section 4.

When incorporated with the ultrasound transducer 2 the image produced by the probe will contain a shadow, by virtue of the presence of the channel 30. Indeed, the image produced will be substantially identical to that produced by the probe 1. However, the enclosed channel 30 provides the user with an improved guide for the insertion of a needle, and a greater integral strength within the waveguide 29. Supplementary guide markings, shown as partial cross hairs 31, may also be provided on the isosceles prism section 4.

A further alternative embodiment of the ultrasound waveguide 32 is shown in FIG. 11. In this example, the waveguide itself is of the type shown in FIG. 10. However, the waveguide 32 is provided with a needle support structure 33. The support structure 33 includes a support block 34 extending outwardly from the posterior face of the waveguide 32. A bore 35 extends through the support block 34 and the right angled isosceles prism section 4 through to the planar anterior face 6. The bore 35 is oriented orthogonally to the planar anterior face 6 of the waveguide 32.

Within the bore 35 is an internal sterile sheath 36. The sheath 36 provides direct support to a needle 37, and provides a degree of resistance to movement of the needle 37.

In use, the operator 19 identifies the lumbar interspace in the manner described above. The needle 37 can be positioned in the sheath 36 before or during the location process. This allows the operator 19 to align the needle 37 easily, without requiring potentially awkward handling by the probe supporting hand, and avoiding the need to use two hands. When the needle is successfully aligned, it can be inserted into the skin.

Referring now to FIG. 12, an ultrasound waveguide 38 in accordance with a yet further alternative embodiment of the present invention is shown. This embodiment is similar to the embodiment shown in FIGS. 1 and 2 and can be seen to comprise the right angled isosceles prism section 4. However, within this embodiment the arcuate recess 13 is formed directly on a non-hypotenuse face of the prism section 4.

The ultrasound waveguide 38 can be further seen to comprise a channel in the form of a slot 39 extending from the hypotenuse face 10 of the right angled isosceles prism section 4 through to the planar anterior face 6. A rear wall 40 of the slot 39 (i.e. that located opposite to the open side of the slot 39) is orientated substantially perpendicular to the planar anterior face 6. The rear wall 40 takes the form of a V-shaped groove, an apex 41 of which is located furthest from the open side of the slot 39. The sides 42 of the V-shaped groove are designed so as to lie at approximately 45° to the face of the prism section that contains the arcuate recess 13.

The width of the slot 39 is approximately 4mm so that it is wide enough to accommodate an epidural needle of any gauge. This width also gives a degree of freedom to manipulate the needle employed by a user.

When the ultrasound waveguide 38 is incorporated with the ultrasound transducer 2 the image produced by the probe will contain a shadow, by virtue of the presence of the slot 39, in a similar manner to that previously described. However, the incorporation of the V-shaped rear wall 40 has the effect of increasing the quality of the detected ultrasound waves. This occurs because the sides 42 act to reflect the ultrasound waves incident on the slot 39 away from the transducer 2, so as to minimise the effects of backscatter from the slot 39 into the transducer 2.

Referring now to FIGS. 13A and 13B an ultrasound waveguide 43, in accordance with a yet further alternative embodiment is shown. FIG. 13A shows waveguide 43A shaped in accordance with the waveguide 38 of FIG. 12, and FIG. 13B shows waveguide 43B shaped in accordance with the waveguide 3 of FIGS. 1 and 2. The waveguides 43A and 43B are formed of a tissue mimicking material. The tissue mimicking material, such as that used in making ultrasound phantoms, is chosen to have the same physical properties as the target object it is imaging, in this case human tissue. A tissue mimicking material suitable for making the waveguides 43A and 43B, shown in FIGS. 13A and 13B, comprises evaporated milk; agar; distilled water; n-propanol and a few drops of a biological cleansing agent used to prevent algae and bacterial growth. An example of a preparation method used for such making a material can be found in the paper published by Ernest L. Madsen, Gray R. Frank & Fang Dong (Liquid or Solid Ultrasonically Tissue-Mimicking Materials With Very Low Scatter. Ultrasound in Medicine & Biology 1998; 4: 535-542.)

The properties of the material that are important in the selection of the material to be used include the acoustic velocity, the acoustic attenuation and the density.

The acoustic velocity property is an important property as it is important that the distance in the waveguide directly relates to the distance on the ultrasound system screen. Ideally, this means the acoustic velocity of the waveguide should be as close as possible to the tissue velocity of the target object as is set in the ultrasound system (a value which itself is a compromise). However, other velocities might be possible provided the additional distance on the screen, because of the waveguide, is calibrated appropriately.

The acoustic attenuation property is of importance so that any reverberations in the waveguide are damped out sufficiently to prevent artifacts in the image. The degree of attenuation required relates to the overall waveguide design, for instance, if an acoustic absorber is included at the edges of the waveguide. The degree of attenuation required also relates to the acoustic match of the waveguide to the tissue of the target object. If it is well matched to tissue and the acoustic absorber is included around the edges, then reverberations are reduced, and attenuation is a less significant third mechanism.

The density of the material is also important because the acoustic impedance of the waveguide matching to the tissue of the target object is crucial. Acoustic impedance is the product of density and velocity; hence if velocity is a set quantity then density can be used to control the acoustic impedance. Exploitation of this property has some limitations as in many cases changing the material in order to change the velocity often has the side effect of changing the density of the material.

Waveguides 43A and 43B, formed of tissue mimicking material, can be made by being casting in the two part mould 44 shown in FIGS. 14A and 14B.

The tissue mimicking waveguide 43A and 43B exhibit impedance qualities such that no special ultrasound gel is required at the interfaces between the transducer and the waveguide; and the waveguide and the target object, with a thin film of water giving good coupling and effective transmission. As the tissue mimicking waveguide shown is made of a pliable material, support can be provided to the waveguide by a support frame 45 such as that shown in FIG. 15.

However, it is not strictly necessary that the material of the waveguide is pliable. The waveguide material, such as that used in the waveguide 43A and 43B shown in FIGS. 13A and 13B may be pliable because of the other requirements and therefore needs a support frame 45 as shown in FIG. 15. However, other materials may satisfy the acoustic requirements of the waveguide, which are also sufficiently rigid to be self-supporting. Similarly, other materials, which are fluid, are also able to satisfy the acoustic requirements of the waveguide however such materials require to be supplied in a suitable container.

It will be evident that various modifications and improvements could be made to the above-described apparatus and methods within the scope of the invention. For example, alternatively shaped recesses could be employed so as to be configurable with alternative ultrasound probes commonly employed by those skilled in the art. In alternative embodiments the waveguide could comprise an acoustic lens for focussing and directing the ultrasound waves so that alternative image fields are produced. The described waveguides are made from Rexolite, however any alternative material with an acoustic impedance to match that of the target object and which is suitable for guiding ultrasound waves may also be employed. For example the described waveguides may be made from Perspex® and gel or water reflectors if assembled in a resilient enough form. Furthermore, the anterior face of the waveguide need not comprises a substantially planar surface. In an alternative embodiment the prism section may be arranged so as to be slightly proud to the cuboidal section so as to aid coupling and placement of the device with a patient. A matching raised surface would then also be incorporated within the cuboidal section near to the arcuate recess so as to maintain the orientation of the device with respect to the patient.

Various aspects of the present invention provide an ultrasound waveguide that can be quickly and easily incorporated with a standard ultrasound transducer so as to form an improved ultrasound probe. The ultrasound probe is suitable for use in the identification and/or location of anatomical features, and alignment with those features. Used in conjunction with appropriate supplementary apparatus, the probe also provides an image to the operator for assisting with location, identification and alignment.

The apparatus is simple and easy to use, and provides images that are interpretable by an operator quickly and accurately. In particular, the operator need not be a specialised radiologist. An anaesthetist or clinician with other areas of expertise is able to interpret the images with minimal supplementary training. Furthermore, the use of ultrasonography is feasible in everyday practice. Little preparation is required and portable machines are commonplace.

The invention has particular application in locating useable lumbar interspaces for epidural or sub-arachnoidal injection. However, it will be appreciated by those skilled in the art that the methods and apparatus described apply equally to the location or identification of other anatomical features of a patient for any purpose. In relation to the location anatomical features these features can be located with improved accuracy and confidence. Therefore, the use of the guidance techniques described is likely to increase patient's willingness to undergo regional anaesthetic, where this is appropriate.

A particular aspect of the present invention enables the formation of images of the lumbar spine without utilising ionising radiation or strong magnetic fields, which have inherent impracticalities. Neither of these alternative techniques would be appropriate before a lumbar puncture or a spinal anaesthetic, and in pregnant patients could in fact be harmful.

It is envisaged that the invention may reduce the need to subject a patient to general anaesthetic, which may not be suitable in a variety of cases. Obese patients pose the additional difficulty that the spine may not be palpable, whilst elderly patients may have an increased propensity for fusion of spinal processes, and thus a higher likelihood of bone strikes.

Furthermore, it is noted that the described techniques apply equally well to the alignment of catheters, as they do to the direct injection methods described herein.

The foregoing description of the invention has been presented for purposes of illustration and description and is not intended to be exhaustive or to limit the invention to the precise form disclosed. The described embodiments were chosen and described in order to best explain the principles of the invention and its practical application to thereby enable others skilled in the art to best utilise the invention in various embodiments and with various modifications as are suited to the particular use contemplated. Therefore, further modifications or improvements may be incorporated without departing from the scope of the invention herein intended.

1. An ultrasound waveguide for coupling with an ultrasound transducer so as to provide a means for identifying a target area on a target object, the ultrasound waveguide comprising an ultrasound transducer coupling means, a guide means and a positioning means for positioning the guide means in relation to the target area on the target object wherein the guide means is provided with a channel that provides a discontinuity within the guide means that causes a discontinuity in an ultrasound signal emitted by an ultrasound probe and which produces a discontinuity in an image created by the ultrasound signal for identifying the target area of the target object.

2. An ultrasound waveguide as claimed in claim 1 wherein, the positioning means comprises an anterior face contactable with a surface of the target object and a posterior face comprising a reflecting section for reflecting an ultrasound field generated by the ultrasound transducer so as to exit the ultrasound waveguide through the anterior face.

3. An ultrasound waveguide as claimed in claim 2 wherein, the anterior face is planar.

4. An ultrasound waveguide as claimed in claim 1 wherein, the ultrasound transducer coupling means is shaped to receive the ultrasound transducer.

5. An ultrasound waveguide as claimed in claim 1 wherein, the ultrasound transducer coupling means further comprises a fastening means for maintaining an acoustic contact between the ultrasound transducer and the ultrasound transducer coupling means.

6. An ultrasound waveguide as claimed in claim 5 wherein, the fastening means is selected from a group comprising a set of clips, nuts and bolts, a frame, tape and a hollow located within the shaped surface.

7. An ultrasound waveguide as claimed in claim 1 wherein, the ultrasound transducer coupling means is provided with a shaped surface that is shaped to conform to the shape of the ultrasound transducer.

8. An ultrasound waveguide as claimed in claim 7 wherein, the shaped surface is arcuate.

9. An ultrasound waveguide as claimed in claim 1 wherein, the channel is shaped to minimise acoustic artefacts produced by an ultrasound signal.

10. An ultrasound waveguide as claimed in claim 1 wherein an acoustic absorber is included in the channel.

11. An ultrasound waveguide as claimed in claims 1 wherein, the channel extends from the reflecting section of the posterior face through to the anterior face.

12. An ultrasound waveguide as claimed in claim 1 wherein, the channel comprises a recess located on an edge of the positioning means.

13. An ultrasound waveguide as claimed in claim 1 wherein, the channel is enclosed by the positioning means.

14. An ultrasound waveguide as claimed in claim 1 wherein the channel is at least partially defined by a first side wall and a second side wall, the first and second side walls being inclined with respect to the normal to the anterior face such that the channel has a first width at the posterior surface and a second width at the anterior surface.

15. An ultrasound waveguide as claimed in claim 14 wherein, the first width at the posterior surface is greater than the second width at the anterior surface.

16. An ultrasound waveguide as claimed in claim 1 wherein, the channel is further defined by an internal lateral side wall that is parallel to the normal to the anterior surface.

17. An ultrasound waveguide as claimed in claim 16 wherein, the internal side wall comprises a groove the sides of which are non parallel to the shaped surface suitable for receiving the-ultrasound transducer.

18. An ultrasound waveguide as claimed in claim 17 wherein, the groove is V-shaped.

19. An ultrasound waveguide as claimed in claim 1 wherein, the guide means comprise a pair of guide members protruding from the reflecting section of the posterior face.

20. An ultrasound waveguide as claimed in claim 1 wherein, the guide means is adapted to receive a needle.

21. An ultrasound waveguide as claimed in claim 20 wherein, the guide means is sized to allow the needle to be redirected following initial penetration of the target object.

22. An ultrasound waveguide as claimed in claim 1 wherein, the guide means is inhomogeneous such that the acoustic impedance of the guide means is variable.

23. An ultrasound waveguide as claimed in claim 1 wherein, the guide means is provided with layers of material at least some of which have different acoustic impedances.

24. An ultrasound waveguide as claimed in claim 1 wherein, the guide means is made from a material with an acoustic impedance to match that of the target object.

25. An ultrasound waveguide as claimed in claim 24 herein, the material is a tissue mimicking material.

26. An ultrasound waveguide as claimed in claim 1 wherein, the guide means comprises a gel.

27. An ultrasound waveguide as claimed claim 1 wherein, the ultrasound waveguide further comprises a support structure for supporting the guide means.

28. An ultrasound waveguide as claimed in claim 27 wherein, the support structure is used to increase the accuracy of the identification of the target area.

29. An ultrasound waveguide as claimed in claim 27 wherein, the support structure is a shell adapted to enclose the guide means.

30. An ultrasound waveguide as claimed in claim 27 wherein, the support structure is an external frame.

31. An ultrasound waveguide as claimed in claim 27 wherein, the support structure further comprises an acoustic absorber lining.

32. An ultrasound waveguide as claimed in claim 27 wherein, the support structure comprises reinforcing threads extending through the guide means.

33. An ultrasound waveguide as claimed in claim 1 wherein, the ultrasound probe further comprises a sheath that provides a sterile barrier between the ultrasound probe and the target object.

34. An ultrasound waveguide as claimed in claim 33 wherein, the sheath envelops the ultrasound transducer.

35. An ultrasound waveguide as claimed in claim 33 wherein, the sheath envelops both the ultrasound transducer and the ultrasound waveguide.

36. An ultrasound waveguide as claimed in claim 33 wherein, the sheath is integrated directly with the ultrasound waveguide.

37. An ultrasound waveguide as claimed in claim 1 claim wherein, the target object is a human body.

38. An ultrasound waveguide as claimed in claim 37 wherein, the target object is the lumbar region of a human body.

39. An ultrasound probe for identifying a target area on a target object, the ultrasound probe comprising an ultrasound transducer and an ultrasound waveguide as claimed in claim 1.

40. An ultrasound probe as claimed in claim 39 further comprising a display for displaying an image produced in response to a signal generated by the ultrasound probe.

41. An ultrasound probe as claimed in claim 40 wherein, the image enables identification of the target area.

42. An ultrasound probe as claimed in claim 40 wherein, the image displays the location of the target area in relation to the guide means.

43. A method of identifying a target area on a target object, the method comprising the steps of:
positioning an ultrasound probe in relation to the target object, the ultrasound probe having an ultrasound waveguide and guide means coupled to an ultrasound transducer;
displaying an image of the target object;
identifying a target area from said image based on an image artefact created by the guide means; and
positioning the guide means in relation to said target area.

44. A method as claimed in claim 43 wherein, the target object is a human body.

45. A method as claimed in claim 43 wherein, the target object is the lumbar region of a human body.

46. A method as claimed in claim 43 wherein the method includes the additional step of aligning the guide means with the target area.

47. A method as claimed in claim 43 wherein the method includes the further step of positioning a needle within the guide means, such that the needle is positioned with respect to the target area.

48. (canceled)

49. A method as claimed in claim 43 wherein, the method may include the additional step of marking the target area on the target object.

50. A method as claimed in claim 47 wherein, the method includes the additional step of displaying an image of the needle in relation to the target object.

51. A method as claimed in claim 43 wherein the target area is a lumbar interspace of a patient, and the guide means is positioned in relation to said lumbar interspace.

52. A method as claimed in claim 51 wherein, the method includes the additional step of positioning a needle with respect to the guide means, such that the needle is positioned with respect to the-lumbar interspace.

53. A method as claimed in claim 52 wherein, the method may includes the additional step of aligning the guide means with the lumbar interspace.

54. A method as claimed claim 50 wherein, the method includes the additional step of directing the displayed image of the needle towards the target object.

55. A method as claimed in claim 43 wherein, the method includes the additional step of marking a target area corresponding to a lumbar interspace of a patient.

56. A method for inserting a needle into a lumbar interspace of a patient, the method comprising the steps of:
positioning an ultrasound probe in relation to the lumbar region of the body of the patient, the ultrasound probe having an ultrasound waveguide and guide means coupled to an ultrasound transducer;
displaying an image of the lumbar region;
identifying a lumbar interspace from said image;
positioning the guide means in relation to said lumbar interspace based on an image artefact created by the guide means; and
inserting a needle into the lumbar region of the patient via the guide means.

57. A method as claimed in claim 56 wherein, the method includes the additional step of aligning the guide means with the lumbar interspace.

58. A method as claimed in claim 56 wherein, the method includes the additional step of displaying an image of the needle in relation to the target object.

59. A method as claimed in claim 56 wherein the method includes the additional step of marking a target area corresponding to the lumbar interspace.
